(19) United States
(12) Patent Application Publication
(10) Pub. No.: US 2003/0176881 A1
(43) Pub. Date: Sep. 18, 2003

(54) Shaver blade

(76) Inventor: B. Alex Barlev, Irving, TX (US)

Correspondence Address: GENE WARZECHA, LINVATEC CORPORATION, 11311 CONCEPT BOULEVARD, LARGO, FL, 33773

(21) Appl. No.: 10/373,993

(22) Filed: Feb. 25, 2003

Related U.S. Application Data
(60) Provisional application No. 60/359,465, filed on Feb. 26, 2002.

(51) Int. Cl. A61B 17/32

(52) U.S. Cl. 606/170; 606/167

(57) ABSTRACT

In one embodiment, a shaver blade has a clog resistant configuration and in another embodiment it is capable of being bent into a small radius of curvature while still enabling the flexible inner member to be removed.

### CROSS-REFERENCE TO RELATED APPLICATIONS

This application claims the benefit of U.S. Provisional Application No. 60/359,465, filed Feb. 26, 2002.

### FIELD OF THE INVENTION

The present invention relates generally to surgical instruments. More particularly, the invention relates to powered instruments for resecting tissue during surgical procedures. Still more particularly, the invention relates to shaver blades adapted for use during endoscopic surgical procedures.

### BACKGROUND OF THE INVENTION

Endoscopic surgical procedures are performed through natural body openings or artificially created portals through which elongated instruments are passed to perform surgical procedures at a worksite within the body. The term “endoscopic” as used herein is intended to mean all such procedures including but not limited to arthroscopy, laparoscopy, subdermal percutaneous surgery, other minimally invasive surgery, etc. Shaver blades are often used during such procedures to resect tissue at the worksite.

A shaver blade generally comprises an elongated tubular inner member rotatably mounted within a stationary elongated tubular outer member. The outer member has an opening or window provided at its distal end and the inner member has at least one cutting edge provided at its distal end and adapted to engage and resect any tissue extending through the window of the outer member. The proximal end of the outer member is provided with a hub adapted to affix the outer member to a handpiece and the inner member is provided with a hub adapted to engage a motor drive shaft situated within the handpiece. Reciprocation or rotation (either continual or oscillating) of the inner member relative to the outer member will cause the cutting edges to resect, abrade or otherwise treat tissue through the cutting window.

Shaver blades are often used with an aspirating means to aspirate resected tissue through the lumen of the inner member to an evacuation port at the proximal end of the inner member, through suction channels in the handpiece and to a receptacle or waste drain.

Shaver blades can be made in either a straight or curved configuration. The inner and outer members are generally cylindrical and the outer diameter of the inner member and the inner diameter of the outer member are very close to each other to minimize the gap between them and to maintain close tolerances between the cutting edges of the inner member and the window of the outer member. In a bendable or pre-bent configuration, the outer member is bent with a selected radius of curvature and the inner member is sufficiently flexible to adapt to the curvature while still being able to rotate or oscillate to move the cutting edges relative to the window.

Occasionally, the lumen of the inner member becomes clogged with tissue. This often requires the inner member to be cleaned out by being removed from the outer member and having the clog either flushed out with fluid or pushed out with a cleaning rod or the like.

It is accordingly an object of this invention to produce a shaver blade with a minimized tendency to clog with tissue.

In a bent configuration, the flexibility of the inner member is an important parameter because it affects the radius of curvature to which the shaver blade may be made. A smaller radius of curvature requires a highly flexible inner member. However, in forming such tight curves, it often becomes difficult to remove the inner member from the outer member to clean out any clogged tissue.

It is therefore another object of this invention to produce a shaver blade capable of being bent into a small radius of curvature while still enabling the flexible inner member to be removed from the outer member.

### BRIEF DESCRIPTION OF THE DRAWINGS

Various embodiments of the present invention will be discussed with reference to the appended drawings. These drawings depict only illustrative embodiments of the invention and are not to be considered limiting of its scope.

FIG. 1 is a front perspective view of a shaver blade constructed in accordance with the principles of this invention.

FIG. 2 is an enlarged view of the distal end of the shaver blade of FIG. 1.

FIG. 3 is a cross-sectional view of FIG. 1.

FIG. 4 is an enlarged view of the distal end of FIG. 3.

FIG. 5 is a front perspective view of the inner member of the shaver blade shown in FIG. 1.

FIG. 6 is an enlarged view of the distal end of FIG. 5.

FIG. 7 is an enlarged view of the proximal end of FIG. 3.

FIG. 8 is an illustrative alternate embodiment of FIG. 4.

FIG. 9 is an illustrative alternate embodiment of FIG. 7.

FIG. 10 is an illustrative alternate embodiment of FIG. 6.

FIG. 11 is an illustrative alternate embodiment of FIG. 6.

FIG. 12 is an illustrative alternate embodiment of FIG. 6.

### DESCRIPTION OF THE ILLUSTRATED EMBODIMENTS

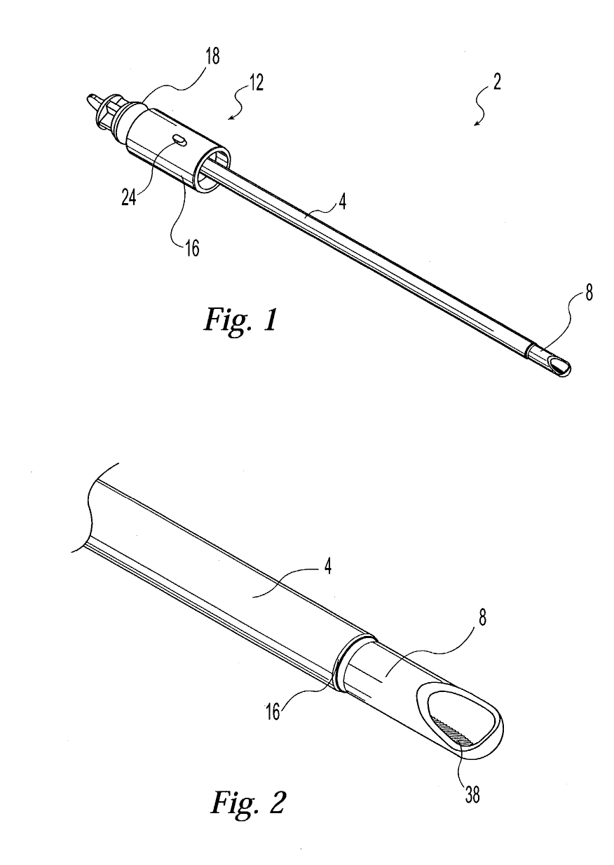

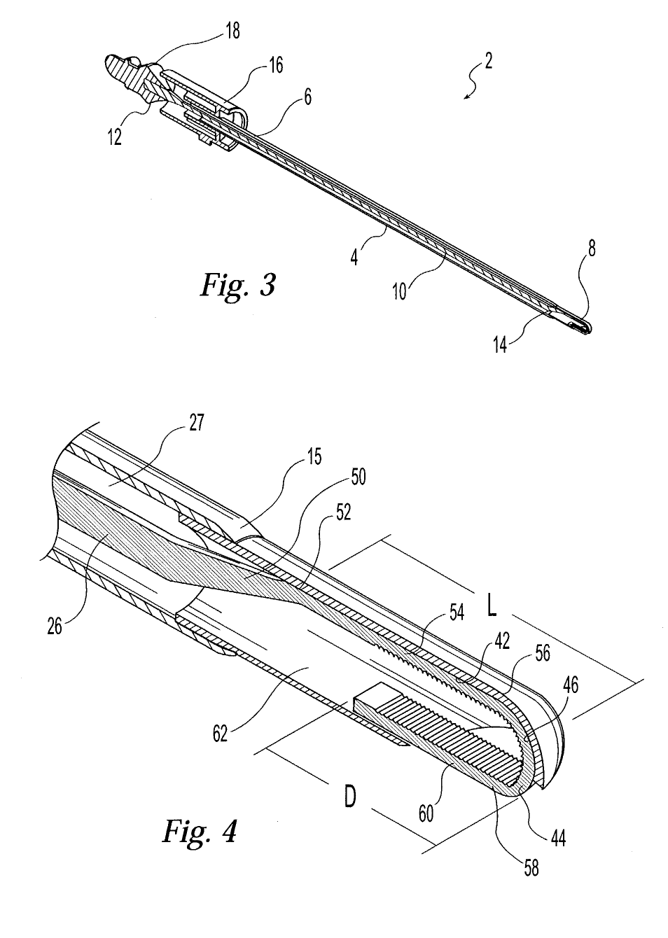

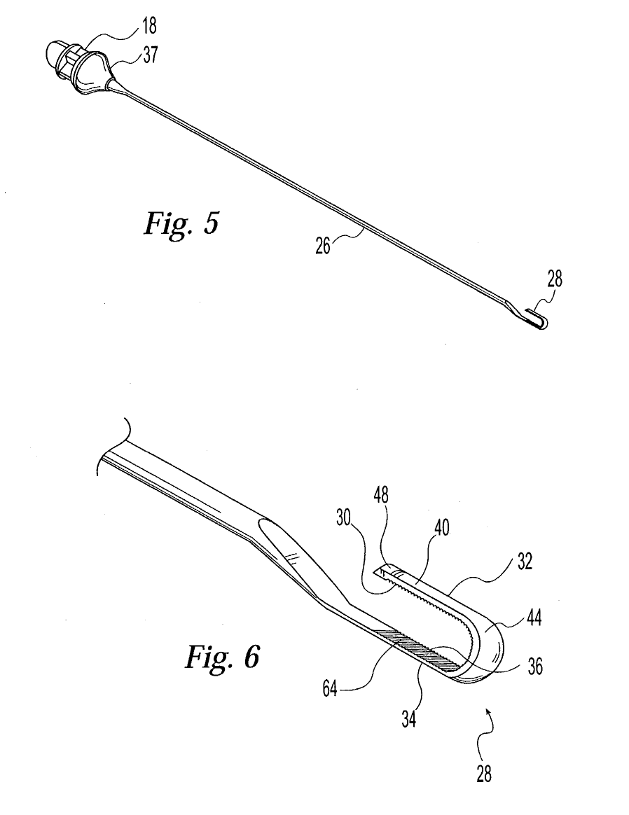

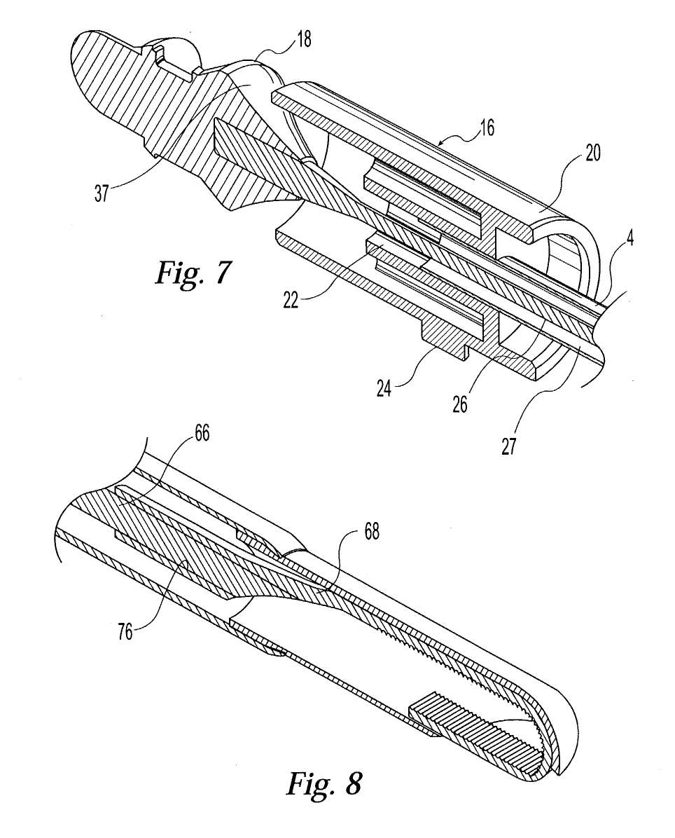

As shown in FIGS. 1-7, an illustrative shaver blade 2, constructed in accordance with the principles of this invention, comprises an elongated outer member 4 having a proximal end 6 and a distal end 8 and an elongated inner member 10 having a proximal end 12 and a distal end 14. The illustrative outer member 4 comprises a hollow tube. The outer member 4 may have a distal end 8 which has a smaller diameter than the main body of the outer member 4. Such a reduced diameter distal end 8 may comprise an integral reduced section, or, as shown in the illustrative embodiment, it may comprise a separate member attached to the main body of the outer member 4. The separate tube may be attached by welding, soldering, crimping, gluing, or other suitable means. A transition section 15 joins the distal end 8 to the main body of the outer member 4. The smaller diameter of the distal end permits the shaver blade 2 to access small spaces while the larger diameter of the main body facilitates the passage of fluid and particles from the distal end 8 to the proximal end 6. At the proximal end 6 of the outer member is a hub 16 and at the proximal end 12 of the inner member is a hub 18. The outer member 4 may be received by a handpiece (not shown) to facilitate relative rotation between the inner 10 and outer 4 members. The outer member 4 may also be received by a handpiece to facilitate relative axial translation between the inner 10 and outer 4 members. In the illustrative embodiment, the hub 16 is attached to the outer member in a torque resisting manner such as a press-fit, welded, soldered, glued, threaded or other suitable joint means. The hub 16 is received by a handpiece in a torque resisting manner. In the illustrative embodiment, the hub comprises a cylindrical body 20 having an axial bore 22 in close fitting relationship to the outer diameter of the outer member 4. The hub 16 has an outer diameter that is received in close fitting relationship within a bore in the handpiece. A key 24 engages a keyway in the handpiece to prevent the hub 16 from rotating relative to the handpiece.

The inner member 10 comprises an elongated shaft 26 extending from its hub 18 to the distal end 14. The inner member 10 may be mounted for translation or rotation relative to the outer member 4. In the illustrative embodiment, the shaft 26 acts to transmit torsional force from the hub to the distal end. The shaft may be of any construction and material that will transmit torque. The illustrative embodiment comprises a solid metal shaft. Exemplary alternative shaft constructs include hollow shafts, twisted filament shafts, cable shafts, and shafts made of polymers. The shaft 26 may be relatively stiff for straight shaver blade applications, or may be made flexible for use in curved shaver blades. The shaft 26 extends coaxially along the middle of the outer member 4 so that an annular passage 27 is formed between the inner 10 and outer 4 members. A cutting member 28 is attached to the distal end 14 of the inner member. The cutting member 28 may be formed as an integral part of the shaft or it may be formed as a separate piece and attached to the shaft such as by welding, soldering, adhesive bonding, crimping, threading, or other suitable attachment means. The illustrative cutting member 28 is formed as an integral extension of the shaft 26 and includes four cutting edges 30, 32, 34, and 36. The outer member 4 includes a cutting window 38 adjacent the cutting member 28. The cutting edges cuts tissue which extends through the window 38. The resected tissue is aspirated proximally through the annular passage 27 between the inner 10 and outer 4 members. The hub 18 attached to the proximal end 12 of the inner member 10 includes a conical diverter surface 37 which diverts aspirated material transversely and into a suction passage within the handpiece. The annular passage 27 presents a relatively large cross section to facilitate the passage of debris and resist clogging. Furthermore, the motion of the inner member 10 tends to dislodge debris that might otherwise form a clog in a simple tube arrangement. In addition, the mass of the inner member 10 is located near its axis and out of contact with the inner wall of the outer member 4 which reduces inertial and frictional loads on the inner member allowing more of the driving force to be delivered to the cutting member 28.

The structure of cutting member 28 is that of a generally “U” shaped, “J” shaped, or partial loop member having an outer surface 40 which is curved to conform to the curvature of the inner cylindrical surface 42 of outer member 4. The illustrative cutting member 28 has an outer surface 40 that is curved circumferentially to conform to the cylindrical inner diameter of the outer member 4. It also has a spherical shaped tip 44 conforming to a like-shaped end 46 of the outer member 4. The tip 44 bears against the end 46 with relatively little resistance to rotation. The free end 48 of the cutting member 28 is bent to provide a desired amount of spring tension to facilitate manufacturing and assembling the shaver blade. The manufacturing tolerances can be relatively generous since the free end can flex inwardly toward the shaft axis as the inner member 10 is inserted into the outer member 4 to create a close fit between the cutting member 28 and the end distal end 8 of the outer member 4. Manufacturing variation in the diameter of the parts is accommodated by the spring tension. The amount of interference, and thus spring tension, between the inner 10 and outer members can be adjusted by bending the free end in or out to provide a close fit without creating too much friction. The illustrative cutting member 28 is attached to the shaft 26 by a transition section 50 extending distally and radially outwardly from its point of attachment to the shaft 26. The distal end of the transition section 50 is joined at a junction 52 with a first straight section 54 which extends distally to a junction 56 with the tip 44. The distance from the transition section 50 to the tip is shown in FIG. 4 as distance L. The tip 44 traverses the axis of the shaver blade and is joined at a junction 58 with a second straight section 60. The second straight section 60 is diametrically opposed to the first straight section 54 and extends proximally to the free end 48 of the cutting member 28. The distance from the tip 44 to the free end 48 is shown in the FIG. 4 as distance D. The free end 48 extends proximally past the cutting window 38 to provide for smooth, snag free rotation of the inner member 10 within the outer member 4. Distance D is less than distance L to provide a large distal passageway 62 for aspirated fluid and debris. The fluid path transitions from the central, distal passageway 62 to the annular passageway 27 to the diverter surface 37 which expands the annular shape into the handpiece. Cleaning of the shaver blade 2 is facilitated by the configuration of the inner member 10. Pulling the inner member 10 out of the outer member 4 causes the cutting member to traverse the entire length of the outer member from the distal end 8 to the proximal end 6. Since the cutting member 28 fits closely within the distal end, any obstruction in the annular passage 27 is pushed out of the outer member 4 ahead of the cutting member 28 as it is pulled along. Once the inner member 10 is removed, it is a simple matter to wipe the shaft 26 and cutting member 28 to remove any adhering debris. There are no interior cavities in the illustrative inner member 10 to trap debris or require further detailed cleaning. The inner member 10 can be reinserted into the outer member 4 and the use of the shaver blade 2 resumed.

The inner member can be rotated in one direction clockwise or counterclockwise. It can also be alternately rotated clockwise and counterclockwise. With single direction rotation, two of the cutting edges; e.g. 30 and 36 or 32 and 34; will cut tissue. This two edge cutting will increase the cutting speed and extend the time that the shaver blade remains sharp over single edge designs. With alternating rotation, all four edges will cut tissue and further extend the blade life. The cutting action of the illustrative shaver blade is alternatively further enhanced by providing teeth 64 or serrations on the cutting edges. The bent tip configuration of the illustrative embodiment facilitates forming serrations across the inner face of the cutting member 28. The serrations can be formed on a straight work piece which is subsequently bent to form the cutting member 28.

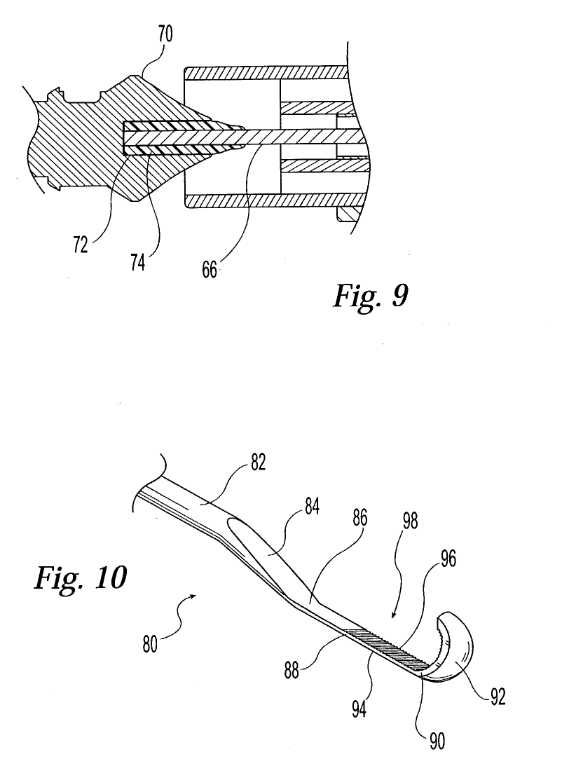

FIGS. 8 and 9 illustrate an alternate embodiment of the shaver blade 2 which is constructed as described above with the exception that a flexible shaft 66 is utilized instead of a solid shaft and a modified cutting member 68 is utilized in order to enable it to be secured to the shaft 66. The shaft 66 may be simply a twisted pair of wires, a cable, a polymeric cylinder, or the like. The hub 70 includes an axial bore 72 receiving the proximal end of the shaft 66. A bonding agent 74; e.g. epoxy, polyurethane, acrylic, etc; fills the space between the shaft 66 and hub 70 to adhere the shaft 66 in the hub 70. The cutting member 68 is a separate piece including an axial bore 76 receiving the distal end of the shaft 66. In the illustrative embodiment, the shaft comprises twisted wires and the cutting member 68 is crimped onto the distal end of the shaft 66.

FIG. 10 illustrates an alternate embodiment of a cutting member 80 for the inner member. The cutting member 80 is attached to the shaft 82 by a transition section 84 extending distally and radially outwardly from its point of attachment to the shaft 82. The distal end of the transition section 84 is joined at a junction 86 with a first straight section 88 which extends distally to a junction 90 joining it to a tip 92. The cutting member 80 terminates at the tip 92 which provides a distal bearing surface. The cutting member 80 includes two cutting edges 94, 96. The bend of the cutting member 80 at the transition section 84, the junction 86, and the junction 90 can be adjusted to provide spring tension to facilitate manufacturing tolerances and assembling the shaver blade. The lack of a second straight section increases the cross-sectional area of the distal fluid path 98 and reduces friction.

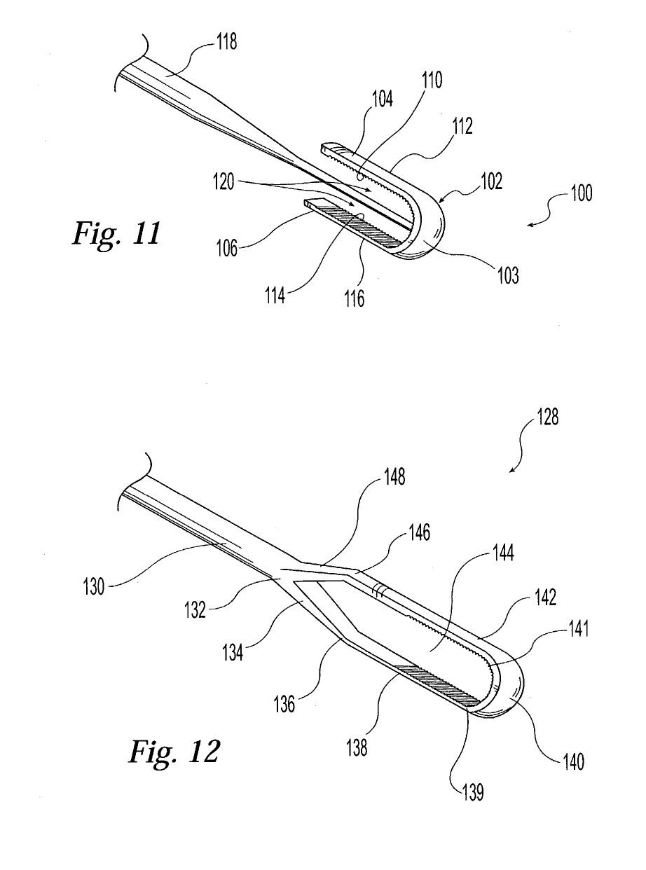

FIG. 11 illustrates an alternate embodiment of a cutting member 100 for the inner member. The cutting member 100 comprises a U-shaped body 102 having a spherical tip 103, first straight member 104 attached to and extending proximally from the tip, and a second straight member 106 attached to and extending proximally from the tip 103 diametrically opposed to the first straight member 104. The cutting member 100 includes four cutting edges 110, 112, 114, and 116. The cutting member is attached to the shaft 118 at the center of the U-shape opposite the spherical tip 103. The cutting member 100 can be formed as an integral part of the shaft 118 or as a separate piece attached using one of the methods listed above for attaching cutting members to shafts. The cutting member 100 provides an annular fluid path 120 from the distal end of the shaver blade to the proximal end of the shaver blade.

FIG. 12 illustrates an alternate embodiment of a cutting member 128 for the inner member. The cutting member 128 comprises a shape similar to the embodiment of FIG. 6 but with an additional portion to close the loop. The illustrative cutting member 128 is attached to the shaft 130 by a junction 132. A first transition section 134 extends distally and radially outwardly from its point of attachment 132 to the shaft 130. The distal end of the transition section 134 is joined at a junction 136 with a first straight section 138 which extends distally to a junction 139 with the tip 140. The tip 140 traverses the axis of the shaver blade and is joined at a junction 141 with a second straight section 142. The second straight section 142 is diametrically opposed to the first straight section 138 and extends proximally. The second straight section 142 is joined at a junction 146 to a second transition section 148. The second transition section 148 extends proximally and radially inwardly from the second straight section 142 to rejoin the shaft at the common junction 132. A central axial fluid path 144 permits aspirant to flow through the cutting member 128 proximally where it transitions to annular flow between the shaft 130 and the outer member.

It will be understood by those skilled in the art that numerous improvements and modifications may be made to the illustrative embodiments of the invention disclosed herein without departing from the spirit and scope thereof.

What is claimed is:

1. A shaver blade comprising:
an elongated tubular outer member having an axis, a proximal end and a distal end, said distal end having an opening;
an elongated inner member having an axis, a proximal end and a distal end, said distal end having a cutting means for cutting material extending into said outer member through said opening, said cutting means comprising a generally U-shaped member having a longitudinally extending cutting edge, said U-shape defined in a plane containing the axis of said inner member.

2. The shaver blade of claim 1 further comprising a generally conically shaped, distally extending diverting surface having an apex and a base, said apex aligned with said axis of said inner member and said base situated proximally thereof.

3. The shaver blade of claim 1 wherein the inner member comprises an elongated shaft and the U-shaped member has a convex side and a concave side, the convex side facing proximally and the shaft attaching to the convex side.

4. A cutting means comprising:
an outer tubular member having an outer window;
an inner member comprising an axially aligned support shaft having a distal end and a proximal end;
a cutting means secured to said distal end of said support shaft, said cutting means comprising a member having, within an axial plane, a J-shaped profile.

5. A shaver blade comprising:
an elongated tubular outer member having a proximal end, a distal end, an axis extending between the proximal and distal end, an outer side wall, an inner side wall, and an inner end wall; and
an inner member having, within an axial plane, a generally elongated partially enclosed loop profile including a body having an outer surface adapted to conform to the inner side surface of said outer member, a distal tip having an outer surface adapted to conform to the inner surface of said inner end wall.

6. The shaver blade of claim 5 wherein the tip comprises a spherical bearing surface.

7. The shaver blade of claim 5 wherein the outer member includes a through window extending from the outer side wall to the inner side wall and the inner member further comprises:
an elongated shaft having a proximal end and a distal end, the partially enclosed loop attached to said distal end of said shaft, said loop adapted to cut tissue extending through said window.

8. The shaver blade of claim 7 wherein the loop further comprises:
a first section attached to said distal end of said shaft and extending distally and radially outwardly therefrom;
a second section attached to said first section and extending distally therefrom, parallel to said axis;
a third section attached to said second section and extending transversely therefrom;
a fourth section attached to said third section and extending proximally therefrom.

9. The shaver blade of claim 8 wherein said fourth section terminates at a point approximately co-lateral with the junction between said first and second sections.

10. The shaver blade of claim 8 wherein the loop further comprises a fifth section attached to said distal end of said shaft and to said fourth section and extending proximally and radially inwardly from said fourth section.

11. The shaver blade of claim 10 wherein the fifth section connects to the shaft to close the loop.

12. A shaver blade comprising:
an elongated tubular outer member having an inside wall with an inside diameter, an outside wall, a proximal end, a distal end, and an axis extending from the proximal end to the distal end;
an elongated inner member having a proximal end, a distal end, and an axis extending from the proximal end to the distal end, the inner member being coaxially mounted for rotation within the outer member, the inner member having a predetermined diameter less than the inside diameter of said outer member, the inner member and outer member defining an annular flow path between them for receiving material flowing proximally through the shaver blade;
a cutting member attached to the distal end of the inner member, the cutting member having at least a first cutting edge offset radially and distally from the distal end of the inner member.

13. The shaver blade of claim 12 wherein the cutting edge is elastically biased radially into contact with the inside wall of the outer member.

14. The shaver blade of claim 12 wherein the outer member includes an opening extending from the outside wall to the inside wall adjacent its distal end, the cutting edge contacting the inside wall adjacent the opening to cut tissue protruding through the opening.

15. The shaver blade of claim 12 wherein the first cutting edge is formed on a first straight section offset radially and distally from the distal end and further comprising a tip connected to the first straight section, distal to the first straight section, and a second straight section connected to the tip and extending proximally from the tip, the second straight section being elastically biased radially into contact with the inside wall of the outer member.

16. The shaver blade of claim 15 wherein the cutting member defines an axial flow path between the first and second straight sections so that material flowing proximally through the shaver blade first traverses an axial flow path and then transitions to an annular flow path.

17. The shaver blade of claim 16 further comprising a conical diverter having a base and an apex, the diverter being coaxially mounted on the inner member adjacent its distal end with the apex facing distally, the diverter able to radially divert material flowing proximally.

18. The shaver blade of claim 12 wherein the cutting edge includes teeth.

19. The shaver blade of claim 12 wherein the teeth comprise serrations formed across a straight section transverse to the axis of the inner member.

20. The shaver blade of claim 12 wherein the inner member comprises a flexible shaft extending between the proximal and distal ends.

21. The shaver blade of claim 20 wherein the shaft comprises an element selected from the list consisting of a cable, twisted wires, and a polymer shaft.

22. The shaver blade of claim 20 wherein the cutting member is attached to the shaft by one of the means of crimping and cementing.

23. A method of making a shaver blade comprising the steps of:
forming an elongate tubular outer member having an inner wall;
forming an elongate inner member, the inner member having a cutting member attached to one end, the cutting member having a cutting edge;
bending the cutting member so that the cutting edge projects axially and distally from the inner member; and
inserting the inner member into the outer member so that the cutting edge is elastically biased into contact with the inner wall of the outer member.

24. The method of claim 23 further comprising the steps of:
serrating the cutting member adjacent the cutting edge transverse to the longitudinal axis of the inner member to form transverse teeth before bending the cutting member.
